(19) United States
(12) Patent Application Publication
(10) Pub. No.: US 2024/0420851 A1
(43) Pub. Date: Dec. 19, 2024

(54) Systems, Methods and Apparatus of a Virtual Medical Masterclass

(71) Applicant: apoQlar GmbH, Hamburg (DE)

(72) Inventor: Sirko Pelzl, Hamburg (DE)

(73) Assignee: apoQlar GmbH, Hamburg (DE)

(21) Appl. No.: 18/337,373

(22) Filed: Jun. 19, 2023

(51) Int. Cl. G16H 80/00; G06Q 10/1093; G06T 7/62; G06T 15/08; G09B 5/06; G09B 5/14; G16H 20/40; G16H 30/20; G16H 40/20

(52) U.S. Cl. CPC G16H 80/00; G06Q 10/1095; G06T 7/62; G06T 15/08; G09B 5/065; G09B 5/14; G16H 20/40; G16H 30/20; G16H 40/20; G06T 2207/10021; G06T 2207/10028; G06T 2207/10136; G06T 2210/41

(57) ABSTRACT

In one aspect, a method of conducting a virtual medical masterclass includes reading a calendar entry from a memory to retrieve from the memory a time/date of a session and to retrieve from the memory an identification of at least one participant involved in the session, retrieving from the memory a plurality of dynamically segmented files, displaying the plurality of patient cases in three-dimensions, the plurality of patient cases being displayed as segmented objects and as volume with natural/cinematic rendering, exchanging a first data to and from medical devices of the medical devices, changing status of operators to allowed interaction with each other to discuss cases and share knowledge, exchanging a second data with a medical application, displaying a 3D point cloud of a live surgery to provide users with a real-life example of surgical techniques, and displaying live stream data from the medical devices.

### FIELD OF THE DISCLOSURE

This disclosure relates generally to a virtual medical masterclass, and more particularly to conducting a virtual medical masterclass.

### BACKGROUND OF THE DISCLOSURE

Training in medical augmented reality has been performed on a one-on-one ad-hoc basis.

### SUMMARY OF THE DISCLOSURE

The above-mentioned shortcomings, disadvantages and problems are addressed herein, which will be understood by reading and studying the following specification.

In one aspect, a method of conducting a virtual medical masterclass includes reading a calendar entry from a memory to retrieve from the memory a time/date of a session and to retrieve from the memory an identification of at least one participant involved in the session, retrieving from the memory a plurality of dynamically segmented files and other data of a plurality of patient cases, displaying on a display device the plurality of patient cases in three-dimensions, the plurality of patient cases being displayed as segmented objects and as volume with natural/cinematic rendering, exchanging a first data to and from medical devices made by different manufacturers of the medical devices to provide a more realistic experience, changing status of operators to allowed interaction with each other to discuss cases and share knowledge, exchanging a second data with a medical application, displaying on the display device a 3D point cloud of a live surgery to provide users with a real-life example of surgical techniques, and displaying live stream data from the medical devices. Virtual medical masterclass participants are able to see how surgeons do a robot surgery, then live 2D or 3D stream is transferred in middle of the virtual medical masterclass, all virtual participants can see the robot screen, works to connect a streamer box to a robot via HDMI

Apparatus, systems, and methods of varying scope are described herein. In addition to the aspects and advantages described in this summary, further aspects and advantages will become apparent by reference to the drawings and by reading the detailed description that follows.

### BRIEF DESCRIPTION OF DRAWINGS

FIG. 1 is a flowchart of a method to conduct a virtual medical masterclass according to an implementation.

FIG. 2 is a block diagram of a conductor of a virtual medical masterclass, according to an implementation.

FIG. 3 is a block diagram of a mixed reality smartglass that conducts a virtual masterclass according to an implementation.

FIG. 4 is a diagram of a configuration of a virtual masterclass, according to an implementation.

FIG. 5 is a block diagram of a virtual masterclass conductor control computer in which different implementations can be practiced.

FIG. 6 is a block diagram of a data acquisition circuit of the virtual masterclass conductor control computer, according to an implementation.

FIG. 7 is a block diagram of a virtual masterclass conductor control hardware and operating environment in which different implementations can be practiced.

FIG. 8 is a block diagram of a virtual masterclass conductor control mobile device, according to an implementation.

### DETAILED DESCRIPTION OF THE DISCLOSURE

In the following detailed description, reference is made to the accompanying drawings that form a part hereof, and in which is shown by way of illustration specific implementations which may be practiced. These implementations are described in sufficient detail to enable those skilled in the art to practice the implementations, and it is to be understood that other implementations may be utilized and that logical, mechanical, electrical and other changes may be made without departing from the scope of the implementations. The following detailed description is, therefore, not to be taken in a limiting sense.

The detailed description is divided into five sections. In the first section, implementations of methods are described. In the second section, apparatus of implementations are described. In the third section, hardware and operating environments in conjunction with which implementations may be practiced are described. Finally, in the fourth section, a conclusion of the detailed description is provided.

A virtual medical masterclass is an advanced training program designed to educate medical professionals on the latest techniques, procedures, and technology. A virtual medical masterclass takes place in a virtual reality environment, providing simulated medical scenarios for participants to practice their skills safely. Taught by experienced doctors and medical experts, the masterclass offers guidance, feedback, and interactive training, enhancing knowledge and skills for better patient care and outcomes. Attendees participate remotely as avatars from anywhere in the world. Medical professionals will be able to experience real medical patient data transformed into 3D holograms during the medical masterclass.

A virtual medical masterclass is designed for experienced senior surgeons interested in using patient-specific 3D models with augmented reality technology for advanced visceral surgery. A virtual medical masterclass covers the latest advances in HoloMedicine and provides practical training for preoperative planning, intraoperative guidance, and postoperative education. Participants learn how to incorporate 3D models into surgical practice to improve patient outcomes. A virtual medical masterclass is ideal for surgeons in an international context who want to stay at the forefront of surgical innovation.

### Method Implementations

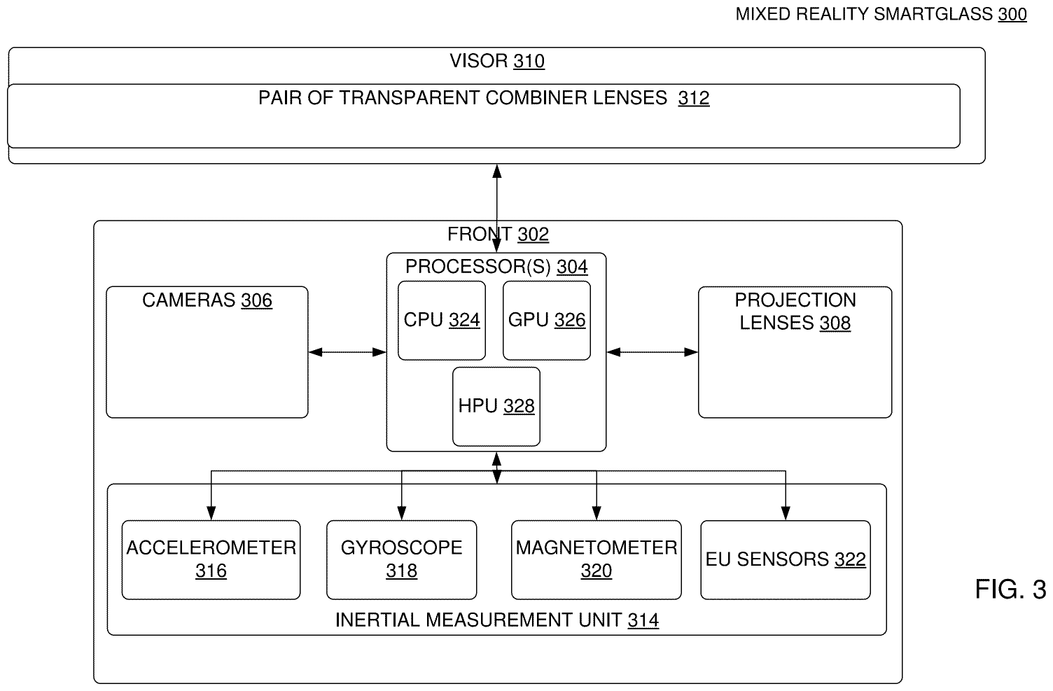

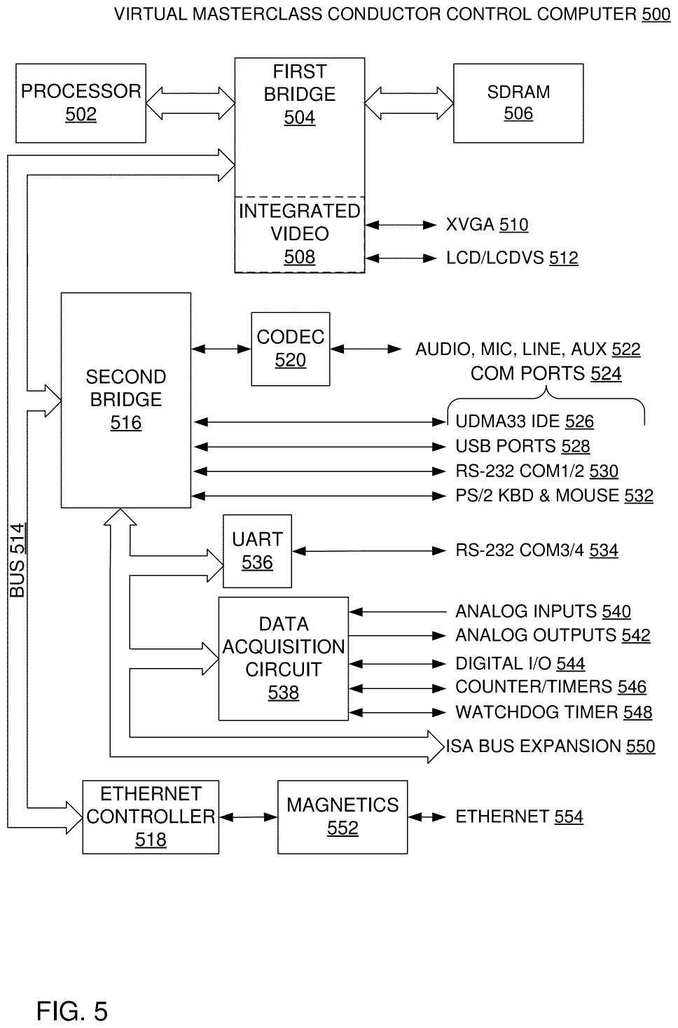

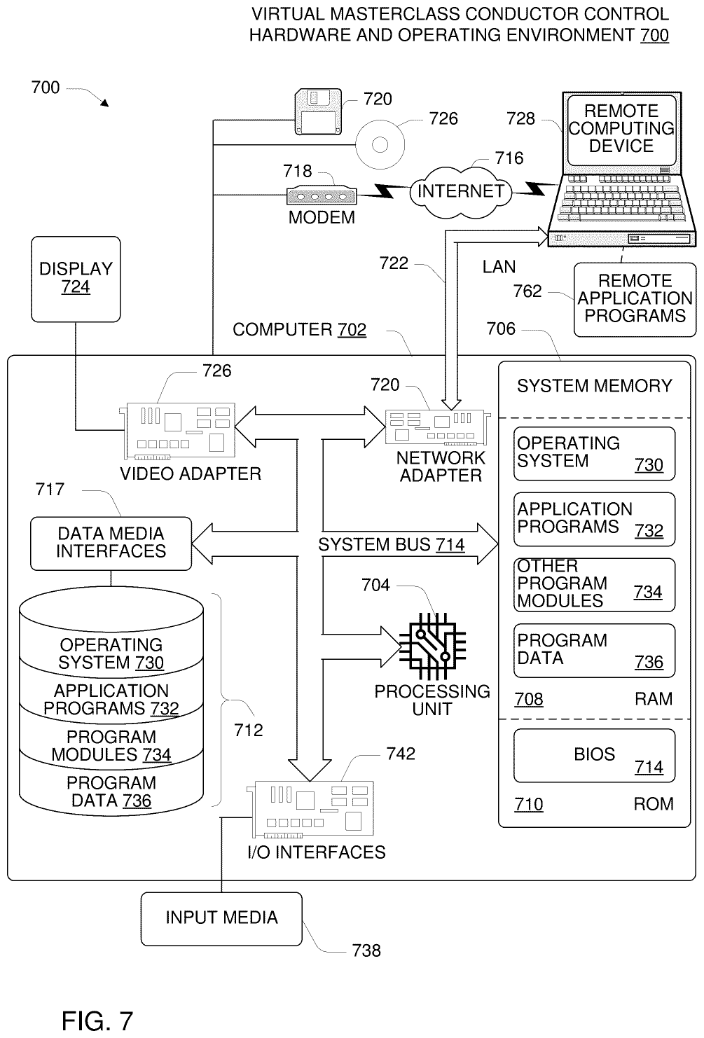

In the previous section, apparatus of the operation of an implementation was described. In this section, the particular methods performed by a processor, such as processor(s) 304 in FIG. 3, processor 502 in FIG. 5, processing unit 704 in FIG. 7 or main processor 802 of such an implementation are described by reference to a series of flowcharts.

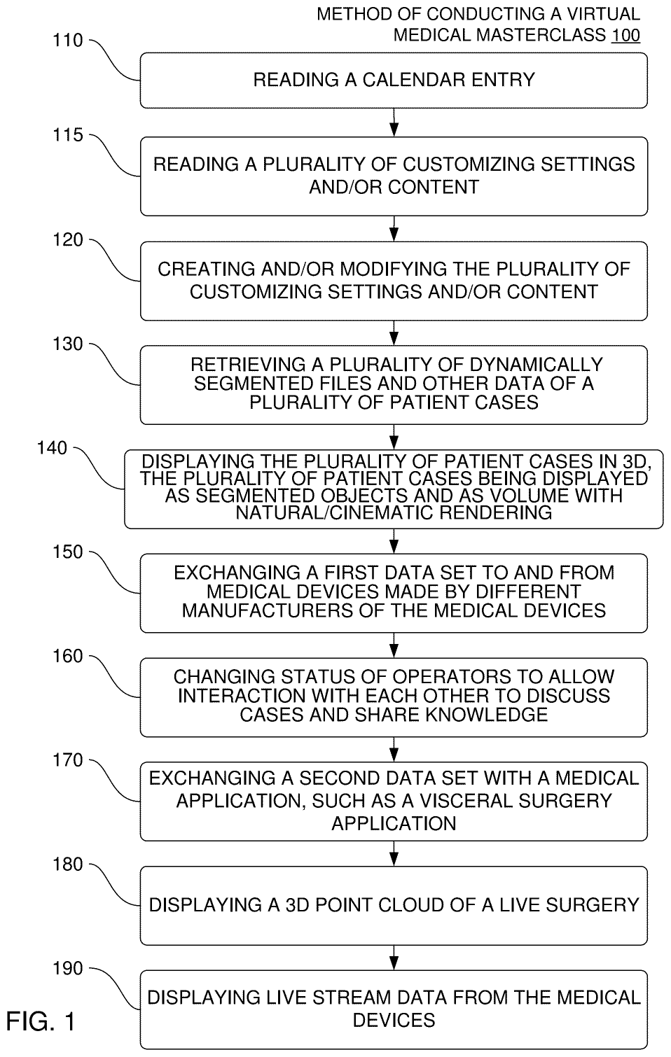

FIG. 1 is a flowchart of a method 100 to conduct a virtual medical masterclass according to an implementation.

In some implementations, method 100 at block 110 includes reading a calendar entry from a memory to retrieve from the memory a time/date of a session and to retrieve from the memory an identification of at least one participant involved in the session. In some implementations of block 110, the calendar entry is retrieved through an online scheduling system.

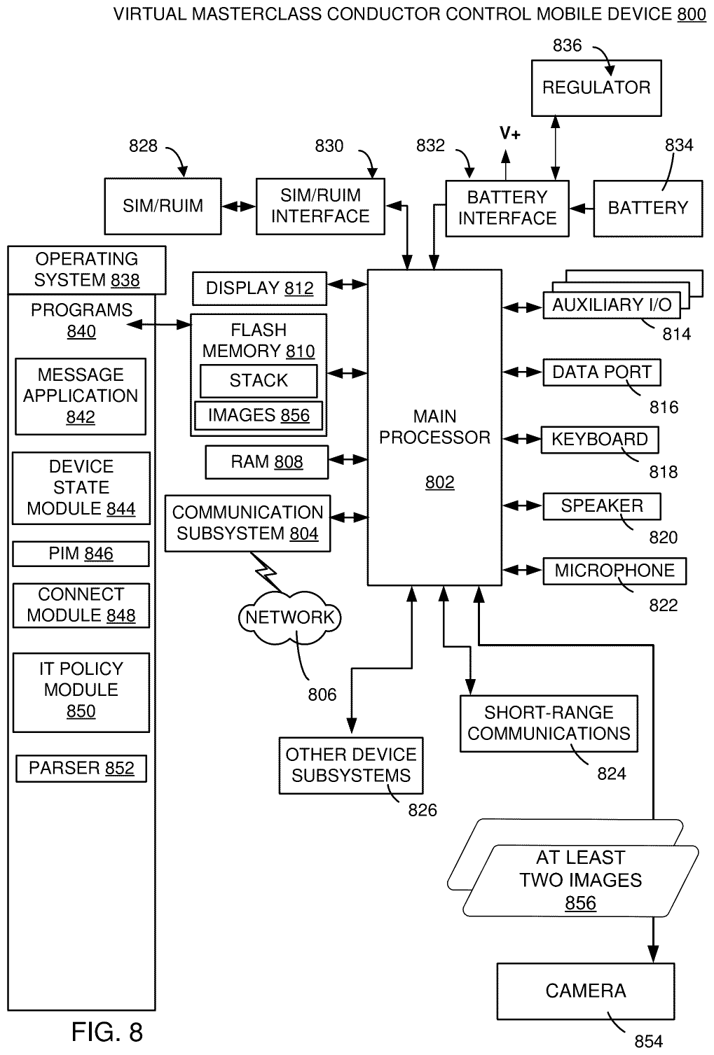

Examples of the memory include SDRAM 506 in FIG. 7, system memory 706 in FIG. 7, flash memory 810 and RAM 808 in FIG. 8.

In some implementations, method 100 at block 115 includes reading a plurality of customizing settings and/or content. The customizing settings include avatars, several room designs, room equipment (mirror to see myself, tables, coffee cup, clock, any other object), presentation slides like PowerPoint, pictures/images, videos, tutorials and the content; all of which can be displayed while the master class is performed in steps 130-190 below, or music to play all of which can be played while the master class is performed in steps 130-190 below, simulations, brand logos, everything to equip a virtual masterclass room. Examples of the content are 3D objects, scenarios and simulations.

In some implementations, method 100 at block 120 also includes further customizing the plurality of customizing settings and/or the content.

In some implementations, method 100 at block 130 includes retrieving from the memory a plurality of dynamically segmented files and other data of a plurality of patient cases. In some implementations of block 130 the dynamically segmented files are downloaded from a secure server to the memory. More specifically, patient-specific data, such as CT scans, MRI scans, etc., can be read and then segmented to show structures or detect tumors, etc.

In some implementations, method 100 at block 140 includes displaying on a display device the plurality of patient cases in three-dimensions, the plurality of patient cases being displayed as segmented objects and as volume with natural/cinematic rendering. Examples of the display device include the mixed reality smartglass 300 in FIG. 3, and XVGA device coupled to the XVGA port 510 or a LCD device operably coupled to the LCD/LCDVS port 512 in FIG. 5 or the display 724 in FIG. 7.

In some implementations, method 100 at block 150 includes exchanging a first data set to and from medical devices made by different manufacturers of the medical devices to provide a more realistic experience. The medical devices and their functions can be presented in the virtual masterclass. One example of the medical devices is a surgical robot. Furthermore, data can be shared to the medical device.

In some implementations, method 100 at block 160 includes changing status of human operators to allow interaction with each other to discuss cases and share knowledge. In some implementations of block 160, the interaction between the operators includes voice and/or video communication. Practical examples of the knowledge that can be shared include multi-disciplinary boards in hospitals to obtain consensus and advise on the best management modality for the patient. In a virtual masterclass a doctor introduces patient cases. Complex Hepatobiliary and Pancreatic cases are often discussed at multi-disciplinary boards in hospitals to obtain consensus and advise on the best management modality for the patient. However, some cases may require input from external experts, or may require a multinational team for consensus and agreement. Traditionally, this would have been done via a virtual call. Though convenient, it may be difficult to convey messages across in a 2D display, and it will be difficult to annotate and appreciate a patient's scan in 3D spatial proximity. Mixed Reality and Virtual Reality (XR) technology has the potential to enhance the immersive experience of users, and display a 3D image of the patient's scan to facilitate discussions. Remote assistance is one of the key features of XR technology. The ability to provide remote assistance goes beyond the simplicity of a video call. It involves transmission of data in real-time both directions, and it needs to be done in an environment that is both user friendly, intuitive, and secure across time and place.

In some implementations, method 100 at block 170 includes exchanging a second data set with a medical application. In some implementations, block 170 exchanging the second data with the medical application further includes exchanging the second data with a visceral surgery application. In some implementations, block 170, exchanging the second data with the medical application, further includes exchanging the second data with a resection planning and volume calculation application to enable users to plan surgeries and calculate a volume of tissue that needs to be removed. In some implementations, block 170 includes resection planning and volume calculation features are specific to a medical field. The shared data can be any specific medical field application—with external planning software etc.

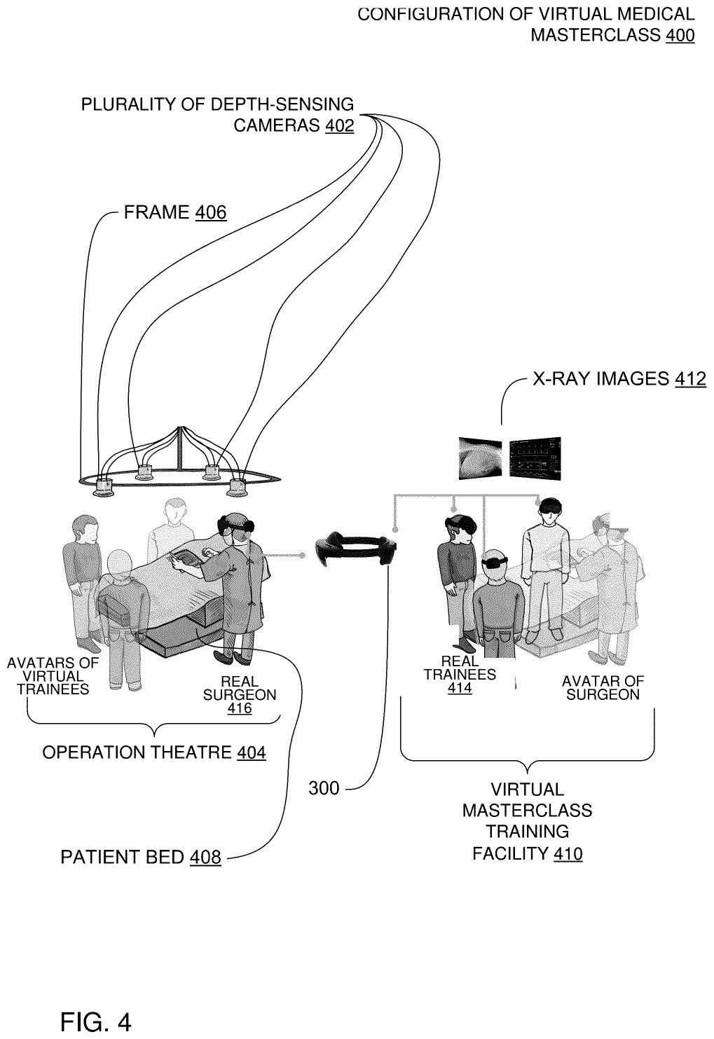

In some implementations, method 100 at block 180 includes displaying on the display device a three-dimensional point cloud and/or a mesh of a live surgery to provide users with a real-life example of surgical techniques. In some implementations of block 180, the three-dimensional point cloud of the live surgery is obtained through a video recording. A use case of block 180 is shown in FIG. 4.

In some implementations, method 100 at block 190 includes displaying live stream data from the medical devices. In some implementations of block 190, medical devices further comprise an ultrasound medical imaging device or a laparoscopic surgical device.

In some implementations, blocks 180 and 190 in method 100 are substituted for displaying on the first display device a live stream data and a three-dimensional point cloud and/or mesh and/or stereoscopic format, any other formats from the medical devices and/or a live surgery scene and/or from a camera mounted on a headset of a second device that is worn by a surgeon and other people to provide users with a real-life example of surgical techniques and live patient view.

Blocks 140, 150, 160, 170, 180 and 190 are performed in reference to the calendar entry that is read from a memory, the time/date of the session and the identification of at least one participant involved in the session of block 110, the plurality of customizing settings and/or content of blocks 115 and 120, and the plurality of dynamically segmented files and other data of a plurality of patient cases of block 130.

In some implementations, during performance of the master class in steps 130-190 above of method 100, an operator can move between to different rooms, such as a room that is designated as being associated with the operator, or a room that is designated as being associated with the host of the master class.

In some implementations, method 100 is implemented as a sequence of computer instructions which, when executed by a processor, such as processor(s) 304 in FIG. 3, processor 502 in FIG. 5, processing unit 704 in FIG. 7 or main processor 802, cause the processor to perform the respective method. In other implementations, method 100 is implemented as a computer-accessible medium having executable instructions capable of directing a processor, such as processor(s) 304 in FIG. 3, processor 502 in FIG. 5, processing unit 704 in FIG. 7 or main processor 802, to perform the respective method. In varying implementations, the medium is a magnetic medium, an electronic medium, or an optical medium.

In some implementations, method 100 can be commercialized to provide a revenue stream to the operator of the method 100, though conventional techniques, such as establishing a paywall around the processor that performs the method 100, in which a user must be authenticated as an authorized user that has paid funds required to operate the method 100 on the processor. The paywall can be either a “hard” paywall that requires a paid subscription by a user before any portion of the virtual masterclass that is performed by the method 100 can be accessed by the user, or the paywall can be either a “soft” paywall that allows the user to access a portion of the virtual masterclass that is performed by the method 100, or allows the user to access all of the virtual masterclass for a limited amount of time, before the user is required to pay the funds.

### Apparatus Implementations

In the previous section, a system level overview of the operation of an implementation was described. In this section, the particular apparatus of such an implementation are described by reference to a series of diagrams.

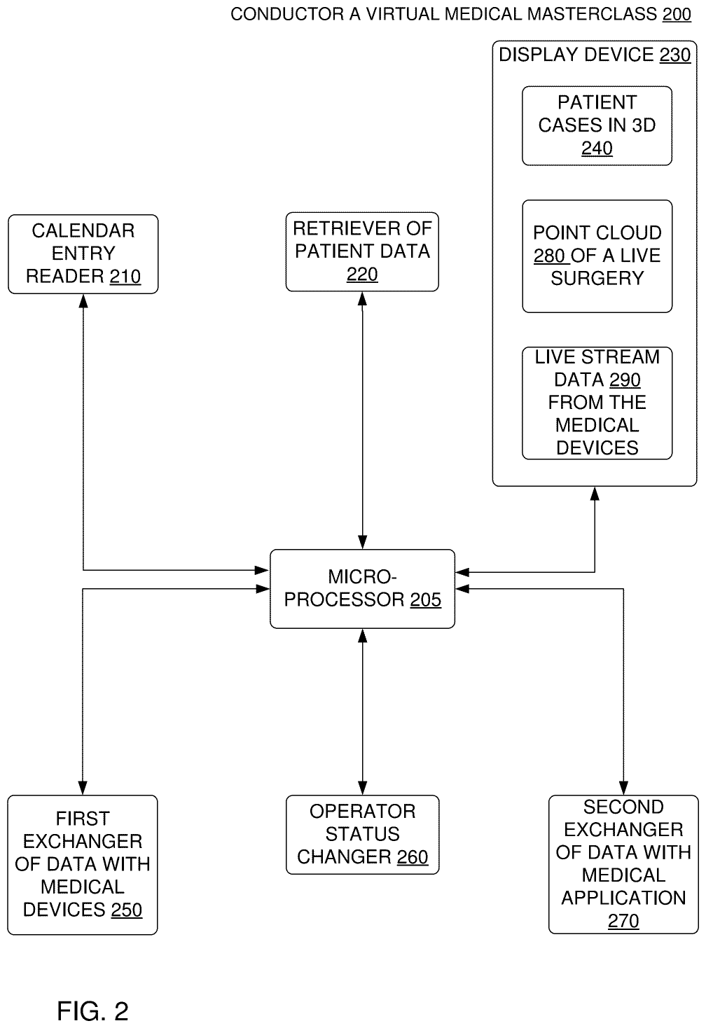

FIG. 2 is a block diagram of a conductor of a virtual medical masterclass 200, according to an implementation.

In some implementations of FIG. 2, the apparatus 200 includes a reader 210 of a calendar entry from a memory to retrieve from the memory a time/date of a session and to retrieve from the memory an identification of at least one participant involved in the session, the reader 210 being operably coupled to the microprocessor 205.

In some implementations of FIG. 2, the apparatus 200 also includes a retriever 220 from the memory a plurality of dynamically segmented files and other data of a plurality of patient cases, the retriever being operably coupled to the microprocessor 205.

In some implementations of FIG. 2, the apparatus 200 also includes a display device 230 that is operable to display the plurality of patient cases 240 in three-dimensions. The plurality of patient cases can be displayed as segmented objects and as volume with natural/cinematic rendering. The display device 230 is operably coupled to the microprocessor 205.

In some implementations of FIG. 2, the apparatus 200 also includes a first exchanger 250 of a first data to and from medical devices made by different manufacturers of the medical devices to provide a more realistic experience. The exchanger 250 of the first data being operably coupled to the microprocessor 205.

In some implementations of FIG. 2, the apparatus 200 also includes an operator status changer 260 that allows interaction with other operators to discuss cases and share knowledge.

The changer 260 being operably coupled to the microprocessor 205.

In some implementations of FIG. 2, the apparatus 200 also includes a second exchanger 270 of a second data with a medical application. The second exchanger 270 of the second data is operably coupled to the microprocessor 205.

In some implementations of FIG. 2, the display device 230 is further operable to display a three-dimensional point cloud 280 of a live surgery to provide users with a real-life example of surgical techniques; and

In some implementations of FIG. 2, the display device 230 is further operable to display live stream data 290 from the medical devices.

In some implementations, apparatus 200 can be commercialized to provide a revenue stream to the operator of the apparatus 200, though conventional techniques, such as establishing a paywall around the processor that performs the apparatus 200, in which a user must be authenticated as an authorized user that has paid funds required to operate the apparatus 200 on the processor. The paywall can be either a “hard” paywall that requires a paid subscription by a user before any portion of the virtual masterclass that is performed by the microprocessor 205 can be accessed by the user, or the paywall can be either a “soft” paywall that allows the user to access a portion of the virtual masterclass that is performed by the microprocessor 205, or allows the user to access all of the virtual masterclass for a limited amount of time, before the user is required to pay the funds.

### Hardware and Operating Environments

FIG. 3 is a block diagram of a mixed reality smartglass 300 that is operable to conduct a virtual medical masterclass, according to an implementation. The mixed reality smartglass 300 can include the apparatus 200 in FIG. 2 and/or can perform the method 100 in FIG. 1. The mixed reality smartglass 300 is a head-mounted display unit and in some implementation is connected to an adjustable, cushioned inner headband, which can tilt the mixed reality smartglass 300 up and down, as well as forward and backward. To wear the unit, the user can fit the mixed reality smartglass 300 on their head, using an adjustment wheel at the back of the headband to secure the mixed reality smartglass 300 around the crown, supporting and distributing the weight of the mixed reality smartglass 300 equally for comfort, before tilting the visor towards the front of the eyes.

In some implementations, the front 302 of the unit houses many of the sensors and related hardware, including the processors 304, cameras 306 and projection lenses 308. In some implementations, the visor 310 is tinted; enclosed in the visor 310 is a pair of transparent combiner lenses 312, in which the projected images are displayed in the lower half. In some implementations, the mixed reality smartglass 300 must be calibrated to the interpupillary distance (IPD), or accustomed vision of the user.

In some implementations, along the bottom edges of the side, located near the user's ears, are a pair of small, 3D audio speakers. The speakers, competing against typical sound systems, do not obstruct external sounds, allowing the user to hear virtual sounds, along with the environment. Using head-related transfer functions, the mixed reality smartglass 300 generates binaural audio, which can simulate spatial effects; meaning the user, virtually, can perceive and locate a sound, as though it is coming from a virtual pinpoint or location.

In some implementations, on the top edge are two pairs of buttons: display brightness buttons above the left ear, and volume buttons above the right ear. Adjacent buttons are shaped differently-one concave, one convex-so that the user can distinguish them by touch.

The mixed reality smartglass 300 includes an inertial measurement unit (IMU) 314 (which includes an accelerometer 316, a gyroscope 318, and a magnetometer 320) four “environment understanding” (EU) sensors 322 (two on each side), and in some implementations, an energy-efficient depth camera with a 120°×120° angle of view, a 2.4-megapixel photographic video camera, a four-microphone array, and an ambient light sensor.

In some implementations, SoC contains a CPU 324 and a GPU 326. In some implementations, the mixed reality smartglass 300 features a custom-made holographic processing unit (HPU) 328, a coprocessor manufactured specifically for the mixed reality smartglass 300. In some implementations, the SoC and the HPU 328 each have 1 GB LPDDR3 and share 8 MB SRAM, with the SoC also controlling 64 GB eMMC and running an operating system. In some implementations, the HPU 328 uses 28 custom DSPs from Tensilica to process and integrates data from the sensors, as well as handling tasks such as spatial mapping, gesture recognition, and voice and speech recognition. In some implementations, the HPU 328 processes “terabytes of information.” The display field of view can be 30°×17.5°. The HPU 2000 in FIG. 20 is one example of the HPU 328.

The mixed reality smartglass 300 can include IEEE 802.11ac Wi-Fi and Bluetooth 4.1 Low Energy (LE) wireless connectivity. The headset can use Bluetooth LE to pair with the included Clicker, a thumb-sized finger-operating input device that can be used for interface scrolling and selecting. The Clicker features a clickable surface for selecting, and an orientation sensor which provides for scrolling functions via tilting and panning of the unit. The Clicker features an elastic finger loop for holding the device, and a USB 2.0 micro-B receptacle for charging its internal battery.

FIG. 4 is a diagram of a configuration of a virtual medical masterclass 400, according to an implementation.

The configuration of a virtual medical masterclass 400 includes an array of a plurality of depth-sensing cameras 402, such as the Microsoft Azure Kinect® depth sensing cameras. The Microsoft Azure Kinect® depth sensing cameras each have color resolution of 4096×3072 px (total 12.6 Mpx), color Field-of-View (H×V) of 90°×59°, depth resolution (WFOV) of 1024×1024 px (1MPx), 512×512 px, (0.26 Mpx), NFOV of 640×576 px (0.37 Mpx), and 320×288 px (0.9 Mpx), depth FOV (H×V) WFO of: 120°×120, NFOV of 75°×65°, depth range WFOV of 0.25 to 2.21 m, 0.25 to 2.88 m, NFOV of 0.50 to 3.86 m and 0.50 to 5.46 m, and an acquisition rate of 30 Hz.

The array of the plurality of depth-sensing cameras 402 is positioned in an operation theater 404 suspended on a custom frame 406 over a bed 408. A surgeon in the operation theatre views a patient on the bed using a mixed reality smartglass, such as the mixed reality smartglass 300.

Trainee surgeons in a virtual masterclass training facility 410 using the mixed reality smartglass will view the patient and X-ray images 412 and vital signs of the patient recorded by an X-Ray system. In the mixed reality smartglass of the surgeon trainees 414, the surgeon trainees 414 will also view the patient on the bed 408 of the X-Ray system and the surgeon 416 in the operation theater 404. In the mixed reality smartglass of the surgeon 416 in the operation theater 404, the surgeon 416 in the operation theater 404 will also view the surgeon trainees 414 in the virtual masterclass training facility 410. In some implementations, rather than an X-ray system, a computed tomography (CT) scanner, a magnetic resonance imaging (MRI) scanner, an ultrasound machine, a positron emission tomography (PET) scanner, a single-photon emission computed tomography (SPECT) scanner, a mammography machine, a fluoroscopy machine, an endoscopy equipment used for internal imaging, an optical coherence tomography (OCT) scanner used for eye imaging, an angiography machine used for imaging blood vessels, nuclear medicine cameras used for various types of imaging, a dental X-ray machine used for dental imaging, a bone densitometer used for measuring bone density, thermography equipment used for imaging body heat patterns, infrared cameras used for thermographic imaging, a digital subtraction angiography (DSA) equipment used for vascular imaging, a C-arm machine used for fluoroscopy during surgeries can be implemented.

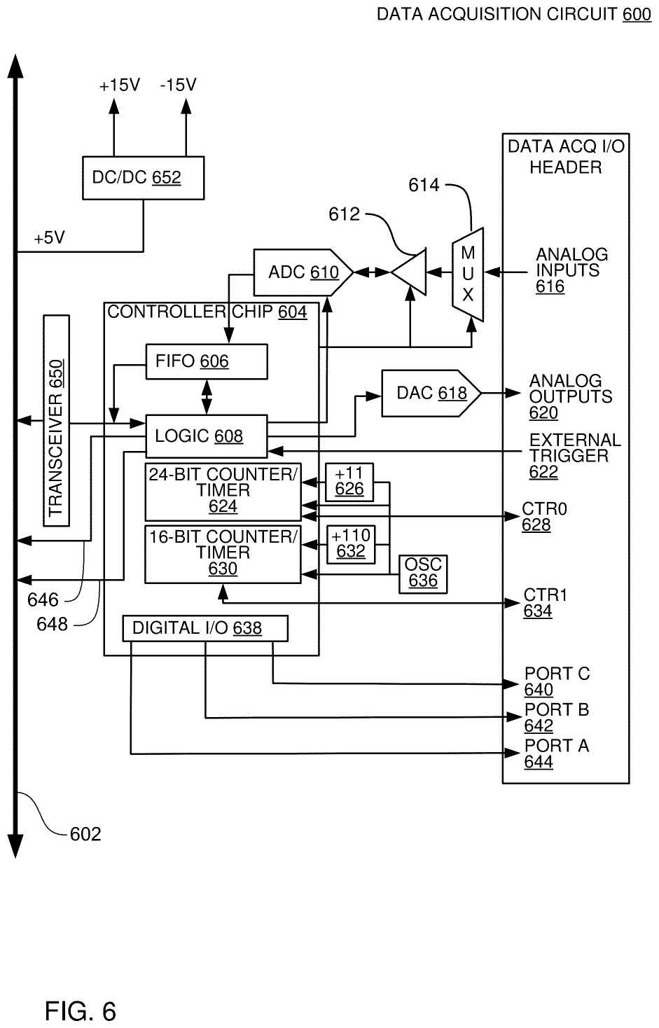

In some implementations of the virtual medical masterclass 400 and the method 100, the amount of time that the trainees 414 spend in the virtual masterclass training facility 410 and the amount of time that the surgeon 416 spends in the operation theater 404 is recorded by the virtual masterclass conductor control computer in FIG. 6, the virtual masterclass conductor control hardware and operating environment 700 and/or the virtual masterclass conductor control mobile device 800. In further implementations, reports are generated to demonstrate conformance with credit point function of continuing medical education (CME) as required by many nations such as Germany, Singapore, China, India, UK and the United States, such as such as EACCME®, issued by the European Accreditation Council for Continuing Medical Education, an Institution of the UEMS, at 24 Rue de l′Industrie, 1040 Brussels, Belgium; the American Medical Association (AMA) at 330 N Wabash Ave, Chicago, IL 60611; or the Royal College of Physicians and Surgeons of Canada at 774 Echo Drive, Ottawa ON Canada K1S 5N8

In some further implementations, a report demonstrating the credit point function is submitted to a medical association by the virtual masterclass conductor control computer in FIG. 6, the virtual masterclass conductor control hardware and operating environment 700 and/or the virtual masterclass conductor control mobile device 800.

FIG. 5 is a block diagram of a virtual masterclass conductor control computer 500 in which different implementations can be practiced. The virtual masterclass conductor control computer 500 includes a processor 502 (such as a Pentium III processor from Intel Corp. in this example) which includes dynamic and static ram and non-volatile program read-only-memory (not shown), a first bridge 504, operating memory 506 (SDRAM in this example). The first bridge 504 includes integrated video 508 that couples the virtual masterclass conductor control computer 500 to a XVGA communication path 510 and a LCD and/or LCDVS device 512.

The first bridge 504 is operably coupled to a bus 514 and the bus 514 is operably coupled to a second bridge 516 and an Ethernet® controller 518.

The second bridge 516 is operably coupled to a CODEC 520 and the CODEC 520 is coupled to an audio port 522. The second bridge 516 is operably coupled to communication ports 524 (e.g., UDMA IDE 526, USB port(s) 528, RS-232 530 COM1/2 and/or keyboard interface 532).

An RS-232 port 534 is coupled through a universal asynchronous receiver/transmitter (UART) 536 to the second bridge 516.

The second bridge 516 is operably coupled to a data acquisition circuit 538 with analog inputs 540 and outputs 542 and digital inputs and outputs 544.

In some implementations of the virtual masterclass conductor control computer 500, the data acquisition circuit 538 is also coupled to counter timer ports 546 and watchdog timer ports 548. In some implementations of the virtual masterclass conductor control computer 500, the second bridge 516 is operably coupled to an expansion bus 550.

In some implementations, the Ethernet® controller 518 is operably coupled to magnetics 552 which is operably coupled to an Ethernet® local area network 554

With proper digital amplifiers and analog signal conditioners, the virtual masterclass conductor control computer 500 can be programmed to drive a display device in apparatus 300 in FIG. 3, either in a predetermined sequence, or interactively modify the combiner lenses 312. Thus the virtual master class can be made available as information that the virtual master class application program can operate on in as part of its decision-making software that acts to conduct the virtual master class.

FIG. 6 is a block diagram of a data acquisition circuit 600 of a virtual masterclass conductor control computer, according to an implementation. The data acquisition circuit 600 is one example of the data acquisition circuit 538 in FIG. 5 above. Some implementations of the data acquisition circuit 600 provide 16-bit A/D performance with input voltage capability up to +/−10V, and programmable input ranges.

The data acquisition circuit 600 can include a bus 602, such as a conventional PC/104 bus. The data acquisition circuit 600 can be operably coupled to a controller chip 604. Some implementations of the controller chip 604 include an analog/digital first-in/first-out (FIFO) buffer 606 that is operably coupled to controller logic 608. In some implementations of the data acquisition circuit 600, the FIFO 606 receives signal data from and analog/digital converter (ADC) 610, which exchanges signal data with a programmable gain amplifier 612, which receives data from a multiplexer 614, which receives signal data from analog inputs 616.

In some implementations of the data acquisition circuit 600, the controller logic 608 sends signal data to the ADC 610 and a digital/analog converter (DAC) 618. The DAC 618 sends signal data to analog outputs. The analog outputs, after proper amplification, can be used to conduct the virtual master class. In some implementations of the data acquisition circuit 600, the controller logic 608 receives signal data from an external trigger 622.

In some implementations of the data acquisition circuit 600, the controller chip 604 includes a digital input/output (I/O) component 638 that sends digital signal data to computer output ports.

In some implementations of the data acquisition circuit 600, the controller logic 608 sends signal data to the bus 602 via a control line 646 and an interrupt line 648. In some implementations of the data acquisition circuit 600, the controller logic 608 exchanges signal data to the bus 602 via a transceiver 650.

Some implementations of the data acquisition circuit 600 include 12-bit D/A channels, programmable digital I/O lines, and programmable counter/timers. Analog circuitry can be placed away from the high-speed digital logic to ensure low-noise performance for important applications. Some implementations of the data acquisition circuit 600 are fully supported by operating systems that can include, but are not limited to, DOS™, Linux™, RTLinux™, QNX™, Windows 98/NT/2000/XP/CE™, Forth™, and VxWorks™ to simplify application development.

FIG. 7 is a block diagram of a virtual masterclass conductor control hardware and operating environment 700 in which different implementations can be practiced. The description of FIG. 7 provides an overview of computer hardware and a suitable computing environment in conjunction with which some implementations can be implemented. Implementations are described in terms of a computer executing computer-executable instructions. However, some implementations can be implemented entirely in computer hardware in which the computer-executable instructions are implemented in read-only memory. Some implementations can also be implemented in client/server computing environments where remote devices that perform tasks are linked through a communications network. Program modules can be located in both local and remote memory storage devices in a distributed computing environment.

Computer 702 includes a processing unit 704, commercially available from Intel, Motorola, Cyrix and others. The computer 702 is one implementation of virtual masterclass conductor control computer 195 in FIG. 1 and computer 206 in FIG. 2. The computer 702 also includes system memory 706 that includes random-access memory RAM 708 and read-only memory ROM 710. The computer 702 also includes one or more mass storage devices 712; and a system bus 714 that operatively couples various system components to the processing unit 704. The RAM 708 and ROM 710, and mass storage devices 712, are types of computer-accessible media. Mass storage devices 712 are more specifically types of nonvolatile computer-accessible media and can include one or more hard disk drives, floppy disk drives, optical disk drives, and tape cartridge drives. The processing unit 704 executes computer programs stored on the computer-accessible media.

Computer 702 can be communicatively connected to the Internet 716 via a communication device, such as modem 718. Internet 716 connectivity is well known within the art. In one implementation, the modem 718 responds to communication drivers to connect to the Internet 716 via what is known in the art as a “dial-up connection.” In another implementation, the communication device is an Ethernet® or network adapter 720 connected to a local-area network (LAN) 722 that itself is connected to the Internet 716 via what is known in the art as a “direct connection” (e.g., T1 line, etc.).

A user enters commands and information into the computer 702 through input devices such as a keyboard (not shown) or a pointing device (not shown). The keyboard permits entry of textual information into computer 702, as known within the art, and implementations are not limited to any particular type of keyboard. Pointing device permits the control of the screen pointer provided by a graphical user interface (GUI) of operating systems such as versions of Microsoft Windows®. Implementations are not limited to any particular pointing device. Such pointing devices include mice, touch pads, trackballs, remote controls and point sticks. Other input devices (not shown) can include a microphone, joystick, game pad, satellite dish, scanner, or the like.

In some implementations, computer 702 is operatively coupled to a display device 724. Display device 724 is connected to the system bus 714 through a video adapter 726. Display device 724 permits the display of information, including computer, video and other information, for viewing by a user of the computer. Implementations are not limited to any particular display device 724. Such display devices include cathode ray tube (CRT) displays (monitors), as well as flat panel displays such as liquid crystal displays (LCD's). In addition to a monitor, computers typically include other peripheral input/output devices such as printers (not shown). Speakers (not shown) provide audio output of signals. Speakers are also connected to the system bus 714.

Computer 702 can be operated using at least one operating system to provide a graphical user interface (GUI) including a user-controllable pointer. Computer 702 can have at least one web browser application program executing within at least one operating system, to permit users of computer 702 to access intranet or Internet world-wide-web pages as addressed by Universal Resource Locator (URL) addresses. Examples of browser application programs include Netscape Navigator® and Microsoft Internet Explorer®.

The computer 702 can operate in a networked environment using logical connections to one or more remote computers, such as remote computer 728. These logical connections are achieved by a communication device coupled to, or a part of, the computer 702. Implementations are not limited to a particular type of communications device. The remote computer 728 can be another computer, a server, a router, a network PC, a client, a peer device or other common network node. The logical connections depicted in FIG. 7 include the local-area network (LAN) 722 and a wide-area network (WAN). Such networking environments are commonplace in offices, enterprise-wide computer networks, intranets and the Internet.

When used in a LAN-networking environment, the computer 702 and remote computer 728 are connected to the local network 722 through network interfaces or adapters 720, which is one type of communications device 718. When used in a conventional WAN-networking environment, the computer 702 and remote computer 728 communicate with a WAN through modems. The modems, which can be internal or external, is connected to the system bus 714. In a networked environment, program modules depicted relative to the computer 702, or portions thereof, can be stored in the remote computer 728.

Computer 702 also includes an operating system 730 that can be stored on the RAM 708 and ROM 710, and/or mass storage device 712, and is and executed by the processing unit 704. Examples of operating systems include Microsoft Windows®, Apple MacOS®, Linux®, UNIX®, providing capability for supporting application programs 732 using, for example, code modules written in the C++® computer programming language. Examples are not limited to any particular operating system, however, and the construction and use of such operating systems are well known within the art.

Instructions can be stored via the mass storage devices 712 or system memory 706, including one or more application programs 732, other program modules 734 and program data 736.

Computer 702 also includes power supply. Each power supply can be a battery.

Some implementations include computer instructions to conduct the virtual master class that can be implemented in instructions or the instructions stored via the mass storage devices 712 or system memory 706 in FIG. 7.

FIG. 8 is a block diagram of a virtual masterclass conductor control mobile device 800, according to an implementation. The virtual masterclass conductor control mobile device 800 includes a number of components such as a main processor 802 that controls the overall operation of the virtual masterclass conductor control mobile device 800. Communication functions, including data and voice communications, are performed through a communication subsystem 804. The communication subsystem 804 receives messages from and sends messages to a wireless network 806. In this exemplary implementation of the virtual masterclass conductor control mobile device 800, the communication subsystem 804 is configured in accordance with the Global System for Mobile Communication (GSM), General Packet Radio Services (GPRS) standards, 3G, 4G, 5G and/or 6G. It will also be understood by persons skilled in the art that the implementations described herein are intended to use any other suitable standards that are developed in the future. The wireless link connecting the communication subsystem 804 with the wireless network 806 represents one or more different Radio Frequency (RF) channels, operating according to defined protocols specified for 4G or 5G communications. With newer network protocols, these channels are capable of supporting both circuit-switched voice communications and packet switched data communications.

Although the wireless network 806 associated with virtual masterclass conductor control mobile device 800 is a GSM/GPRS, 3G, 4G, 5G and/or 6G wireless network in one exemplary implementation, other wireless networks may also be associated with the virtual masterclass conductor control mobile device 800 in variant implementations. The different types of wireless networks that may be employed include, for example, data-centric wireless networks, voice-centric wireless networks, and dual-mode networks that can support both voice and data communications over the same physical base stations. Combined dual-mode networks include, but are not limited to, Code Division Multiple Access (CDMA) or CDMA2000 networks, GSM/GPRS networks, 3G, 4G, 5G and/or 6G. Some other examples of data-centric networks include WiFi 802.11, Mobitex™ and DataTAC™ network communication systems. Examples of other voice-centric data networks include Personal Communication Systems (PCS) networks like GSM and Time Division Multiple Access (TDMA) systems.

The main processor 802 also interacts with additional subsystems such as a Random Access Memory (RAM) 808, a flash memory 810, a display 812, an auxiliary input/output (I/O) subsystem 814, a data port 816, a keyboard 818, a speaker 820, a microphone 822, short-range communications 824 and other device subsystems 826.

Some of the subsystems of the virtual masterclass conductor control mobile device 800 perform communication-related functions, whereas other subsystems may provide “resident” or on-device functions. By way of example, the display 812 and the keyboard 818 may be used for both communication-related functions, such as entering a text message for transmission over the wireless network 806, and device-resident functions such as a calculator or task list.

The virtual masterclass conductor control mobile device 800 can send and receive communication signals over the wireless network 806 after required network registration or activation procedures have been completed. Network access is associated with a subscriber or user of the virtual masterclass conductor control mobile device 800. To identify a subscriber, the virtual masterclass conductor control mobile device 800 requires a SIM/RUIM card 828 (i.e. Subscriber Identity Module or a Removable User Identity Module) to be inserted into a SIM/RUIM interface 830 in order to communicate with a network. The SIM card or RUIM 828 is one type of a conventional “smart card” that can be used to identify a subscriber of the virtual masterclass conductor control mobile device 800 and to customize the virtual masterclass conductor control mobile device 800, among other aspects. Without the SIM card 828, the virtual masterclass conductor control mobile device 800 is not fully operational for communication with the wireless network 806. By inserting the SIM card/RUIM 828 into the SIM/RUIM interface 830, a subscriber can access all subscribed services. Services may include: web browsing and messaging such as e-mail, voice mail, Short Message Service (SMS), and Multimedia Messaging Services (MMS). More advanced services may include: point of sale, field service and sales force automation. The SIM card/RUIM 828 includes a processor and memory for storing information. Once the SIM card/RUIM 828 is inserted into the SIM/RUIM interface 830, it is coupled to the main processor 802. In order to identify the subscriber, the SIM card/RUIM 82 can include some user parameters such as an International Mobile Subscriber Identity (IMSI). An advantage of using the SIM card/RUIM 828 is that a subscriber is not necessarily bound by any single physical mobile device. The SIM card/RUIM 828 may store additional subscriber information for a mobile device as well, including datebook (or calendar) information and recent call information. Alternatively, user identification information can also be programmed into the flash memory 810.

The virtual masterclass conductor control mobile device 800 is a battery-powered device and includes a battery interface 832 for receiving one or more rechargeable batteries 834. In one or more implementations, the battery 834 can be a smart battery with an embedded microprocessor. The battery interface 832 is coupled to a regulator 836, which assists the battery 834 in providing power V+ to the virtual masterclass conductor control mobile device 800. Although current technology makes use of a battery, future technologies such as micro fuel cells may provide the power to the virtual masterclass conductor control mobile device 800.

The virtual masterclass conductor control mobile device 800 also includes an operating system 838 and software components 840 to 852 which are described in more detail below. The operating system 838 and the software components 840 to 852 that are executed by the main processor 802 are typically stored in a persistent store such as the flash memory 810, which may alternatively be a read-only memory (ROM) or similar storage element (not shown). Those skilled in the art will appreciate that portions of the operating system 838 and the software components 840 to 852, such as specific device applications, or parts thereof, may be temporarily loaded into a volatile store such as the RAM 808. Other software components can also be included.

The subset of software components 840 that control basic device operations, including data and voice communication applications, will normally be installed on the virtual masterclass conductor control mobile device 800 during its manufacture. Other software applications include a message application 842 that can be any suitable software program that allows a user of the virtual masterclass conductor control mobile device 800 to send and receive electronic messages. Various alternatives exist for the message application 842 as is well known to those skilled in the art. Messages that have been sent or received by the user are typically stored in the flash memory 810 of the virtual masterclass conductor control mobile device 800 or some other suitable storage element in the virtual masterclass conductor control mobile device 800. In one or more implementations, some of the sent and received messages may be stored remotely from the virtual masterclass conductor control mobile device 800 such as in a data store of an associated host system with which the virtual masterclass conductor control mobile device 800 communicates.

The software applications can further include a device state module 844, a Personal Information Manager (PIM) 846, and other suitable modules (not shown). The device state module 844 provides persistence, i.e. the device state module 845 ensures that important device data is stored in persistent memory, such as the flash memory 810, so that the data is not lost when the virtual masterclass conductor control mobile device 800 is turned off or loses power.

The PIM 846 includes functionality for organizing and managing data items of interest to the user, such as, but not limited to, e-mail, contacts, calendar events, voice mails, appointments, and task items. A PIM application has the ability to send and receive data items via the wireless network 806. PIM data items may be seamlessly integrated, synchronized, and updated via the wireless network 806 with the mobile device subscriber's corresponding data items stored and/or associated with a host computer system. This functionality creates a mirrored host computer on the virtual masterclass conductor control mobile device 800 with respect to such items. This can be particularly advantageous when the host computer system is the mobile device subscriber's office computer system.

The virtual masterclass conductor control mobile device 800 also includes a connect module 848, and an IT policy module 850. The connect module 848 implements the communication protocols that are required for the virtual masterclass conductor control mobile device 800 to communicate with the wireless infrastructure and any host system, such as an enterprise system, with which the virtual masterclass conductor control mobile device 800 is authorized to interface. Examples of a wireless infrastructure and an enterprise system are given in FIG. 4-8, which are described in more detail below.

The connect module 848 includes a set of APIs that can be integrated with the virtual masterclass conductor control mobile device 800 to allow the virtual masterclass conductor control mobile device 800 to use any number of services associated with the enterprise system. The connect module 848 allows the virtual masterclass conductor control mobile device 800 to establish an end-to-end secure, authenticated communication pipe with the host system. A subset of applications for which access is provided by the connect module 848 can be used to pass IT policy commands from the host system to the virtual masterclass conductor control mobile device 800. This can be done in a wireless or wired manner. These instructions can then be passed to the IT policy module 850 to modify the configuration of the virtual masterclass conductor control mobile device 800. Alternatively, in some cases, the IT policy update can also be done over a wired connection.

The IT policy module 850 receives IT policy data that encodes the IT policy. The IT policy module 850 then ensures that the IT policy data is authenticated by the virtual masterclass conductor control mobile device 800. The IT policy data can then be stored in the flash memory 810 in its native form. After the IT policy data is stored, a global notification can be sent by the IT policy module 850 to all of the applications residing on the virtual masterclass conductor control mobile device 800. Applications for which the IT policy may be applicable then respond by reading the IT policy data to look for IT policy rules that are applicable.

The IT policy module 850 can include a parser 852, which can be used by the applications to read the IT policy rules. In some cases, another module or application can provide the parser. Grouped IT policy rules, described in more detail below, are retrieved as byte streams, which are then sent (recursively) into the parser to determine the values of each IT policy rule defined within the grouped IT policy rule. In one or more implementations, the IT policy module 850 can determine which applications are affected by the IT policy data and send a notification to only those applications. In either of these cases, for applications that are not being executed by the main processor 802 at the time of the notification, the applications can call the parser or the IT policy module 850 when they are executed to determine if there are any relevant IT policy rules in the newly received IT policy data.

All applications that support rules in the IT Policy are coded to know the type of data to expect. For example, the value that is set for the “WEP User Name” IT policy rule is known to be a string; therefore the value in the IT policy data that corresponds to this rule is interpreted as a string. As another example, the setting for the “Set Maximum Password Attempts” IT policy rule is known to be an integer, and therefore the value in the IT policy data that corresponds to this rule is interpreted as such.

After the IT policy rules have been applied to the applicable applications or configuration files, the IT policy module 850 sends an acknowledgement back to the host system to indicate that the IT policy data was received and successfully applied.

Other types of software applications can also be installed on the virtual masterclass conductor control mobile device 800. These software applications can be third party applications, which are added after the manufacture of the virtual masterclass conductor control mobile device 800. Examples of third party applications include games, calculators, utilities, etc.

The additional applications can be loaded onto the virtual masterclass conductor control mobile device 800 through at least one of the wireless network 806, the auxiliary I/O subsystem 814, the data port 816, the short-range communications subsystem 824, or any other suitable device subsystem 824. This flexibility in application installation increases the functionality of the virtual masterclass conductor control mobile device 800 and may provide enhanced on-device functions, communication-related functions, or both. For example, secure communication applications may enable electronic commerce functions and other such financial transactions to be performed using the virtual masterclass conductor control mobile device 800.

The data port 816 enables a subscriber to set preferences through an external device or software application and extends the capabilities of the virtual masterclass conductor control mobile device 800 by providing for information or software downloads to the virtual masterclass conductor control mobile device 800 other than through a wireless communication network. The alternate download path may, for example, be used to load an encryption key onto the virtual masterclass conductor control mobile device 800 through a direct and thus reliable and trusted connection to provide secure device communication.

The data port 816 can be any suitable port that enables data communication between the virtual masterclass conductor control mobile device 800 and another computing device. The data port 816 can be a serial or a parallel port. In some instances, the data port 816 can be a USB port that includes data lines for data transfer and a supply line that can provide a charging current to charge the battery 834 of the virtual masterclass conductor control mobile device 800.

The short-range communications subsystem 824 provides for communication between the virtual masterclass conductor control mobile device 800 and different systems or devices, without the use of the wireless network 806. For example, the subsystem 824 may include an infrared device and associated circuits and components for short-range communication. Examples of short-range communication standards include standards developed by the Infrared Data Association (IrDA), Bluetooth, and the 802.11 family of standards developed by IEEE.

In use, a received signal such as a text message, an e-mail message, or web page download will be processed by the communication subsystem 804 and input to the main processor 802. The main processor 802 will then process the received signal for output to the display 812 or alternatively to the auxiliary I/O subsystem 814. A subscriber may also compose data items, such as e-mail messages, for example, using the keyboard 818 in conjunction with the display 812 and possibly the auxiliary I/O subsystem 814. The auxiliary subsystem 814 may include devices such as: a touch screen, mouse, track ball, infrared fingerprint detector, or a roller wheel with dynamic button pressing capability. The keyboard 818 is preferably an alphanumeric keyboard and/or telephone-type keypad. However, other types of keyboards may also be used. A composed item may be transmitted over the wireless network 806 through the communication subsystem 804.

For voice communications, the overall operation of the virtual masterclass conductor control mobile device 800 is substantially similar, except that the received signals are output to the speaker 820, and signals for transmission are generated by the microphone 822. Alternative voice or audio I/O subsystems, such as a voice message recording subsystem, can also be implemented on the virtual masterclass conductor control mobile device 800. Although voice or audio signal output is accomplished primarily through the speaker 820, the display 812 can also be used to provide additional information such as the identity of a calling party, duration of a voice call, or other voice call related information.

In some implementations, the virtual masterclass conductor control mobile device 800 includes a camera 854 receiving a plurality of images 856 from and examining pixel-values of the plurality of images 856.

### CONCLUSION

A virtual masterclass conductor is described. A technical effect of the virtual master class is to communicate the latest techniques, procedures, and technology in holographic surgery in reference to three-dimensional point cloud and live-streaming data. Although specific implementations are illustrated and described herein, it will be appreciated by those of ordinary skill in the art that any arrangement which is calculated to achieve the same purpose may be substituted for the specific implementations shown. This application is intended to cover any adaptations or variations. For example, although described in three dimensional terms, one of ordinary skill in the art will appreciate that implementations can be made in two-dimensional or any other form that provides the required function.

In particular, one of skill in the art will readily appreciate that the names of the methods and apparatus are not intended to limit implementations. Furthermore, additional methods and apparatus can be added to the components, functions can be rearranged among the components, and new components to correspond to future enhancements and physical devices used in implementations can be introduced without departing from the scope of implementations. One of skill in the art will readily recognize that implementations are applicable to future electrically conducting dome, different electrically conducting coils and new power sources.

The terminology used in this application meant to include all medical devices and mixed reality smartglasses and alternate technologies which provide the same functionality as described herein.

An apparatus operable to conduct a virtual medical masterclass that includes a microprocessor, a reader of a calendar entry from a memory to retrieve from the memory a time/date of a session and to retrieve from the memory an identification of at least one participant involved in the session, the reader being operably coupled to the microprocessor, a retriever from the memory a plurality of dynamically segmented files and other data of a plurality of patient cases, the retriever being operably coupled to the microprocessor, a display device operable to display the plurality of patient cases in three-dimensions, the plurality of patient cases being displayed as segmented objects and as volume with natural/cinematic rendering, the display device being operably coupled to the microprocessor, an exchanger of a first data to and from medical devices made by different manufacturers of the medical devices to provide a more realistic experience, the exchanger of the first data being operably coupled to the microprocessor, a changer of a status of operators to allowed interaction with each other to discuss cases and share knowledge, the changer being operably coupled to the microprocessor, an exchanger of a second data with a medical application, the exchanger of the second data being operably coupled to the microprocessor, the display device being further operable to display a live stream data and a three-dimensional point cloud of a live surgery and/or mesh and/or stereoscopic format, any other formats from the medical devices and/or a live surgery scene and/or from a camera mounted on a headset of a second device that is worn by a surgeon, and the display device further operable to display live stream data from the medical devices. In some implementations, the calendar entry is retrieved through an online scheduling system. In some implementations, the dynamically segmented files are downloaded from a secure server to the memory. In some implementations, the medical devices include devices from Medtronic. In some implementations, the three-dimensional point cloud of the live surgery is obtained through a video recording. In some implementations, interaction between the operators includes voice and/or video communication.

An apparatus operable to conduct a virtual medical masterclass that includes a microprocessor, a reader being operably coupled to the microprocessor and having computer instructions that when executed read a calendar entry from a memory to retrieve from the memory a time/date of a session and to retrieve from the memory an identification of at least one participant involved in the session, a retriever being operably coupled to the microprocessor and having computer instructions that when executed retrieve from the memory a plurality of dynamically segmented files and other data of a plurality of patient cases, a display device being operably coupled to the microprocessor and having computer instructions that when executed display the plurality of patient cases in three-dimensions, the plurality of patient cases being displayed as segmented objects and as volume with natural/cinematic rendering, a first exchanger being operably coupled to the microprocessor and having computer instructions that when executed exchange a first data to and from medical devices made by different manufacturers of the medical devices to provide a more realistic experience, a changer being operably coupled to the microprocessor and having computer instructions that when executed change a status of operators to allowed interaction with each other to discuss cases and share knowledge, a second exchanger being operably coupled to the microprocessor and having computer instructions that when executed exchange a second data with a medical application, the display device having further computer instructions that when executed being further operable to display a live stream data and a three-dimensional point cloud and/or mesh and/or stereoscopic format, any other formats from the medical devices and/or a live surgery scene and/or from a camera mounted on a headset of a second device that is worn by a surgeon and other people to provide users with a real-life example of surgical techniques and live patient view, and the display device having further computer instructions that when executed being further operable to display live stream data from the medical devices. In some implementations, the apparatus further includes a reader from the memory of content, the reader being operably coupled to the microprocessor. In some implementations, the second exchanger of the second data with the medical application further comprises computer instructions that when executed exchange the second data with a visceral surgery application. In some implementations, the second exchanger of the second data with the medical application further comprises computer instructions that when executed exchange the second data with a general surgery plan application wherein the general surgery plan application further comprises a resection planning and volume calculation application in visceral surgery (hepatectormy) to enable users to plan surgeries and calculate a volume of tissue that needs to be removed. In some implementations, the resection planning and volume calculation features are specific to a medical field. In some implementations, the medical devices further comprise an ultrasound medical imaging device. In some implementations, the medical devices further comprise a laparoscopic surgical device. In some implementations, the display device further comprises first and/or second display device further comprises a XR (Extended reality device) such as mixed reality smartglasses device like Hololens or virtual reality smartglasses like Meta Quest or the combination like Apple Vision Pro and other providers. In some implementations, the reader further comprises computer instructions that when executed retrieve the calendar entry through an online scheduling system. In some implementations, the dynamically segmented files are downloaded from a secure server to the memory. In some implementations, the medical devices include devices from Medtronic. In some implementations, the three-dimensional point cloud of the live surgery is obtained through a video recording. In some implementations, interaction between the operators includes voice and/or video communication.

An apparatus operable to conduct a virtual medical masterclass that includes a microprocessor, a memory being operably coupled to the microprocessor and having computer instructions that when executed, cause a reading of a calendar entry from the memory a time/date of a session and to retrieve from the memory an identification of at least one participant involved in the session, cause a retrieval from the memory a plurality of dynamically segmented files and other data of a plurality of patient cases, cause a display device to display the plurality of patient cases in three-dimensions, the plurality of patient cases being displayed as segmented objects and as volume with natural/cinematic rendering, cause an exchange of a first data to and from medical devices made by different manufacturers of the medical devices to provide a more realistic experience, cause a change of a status of operators to allowed interaction with each other to discuss cases and share knowledge, cause an exchange of a second data with a medical application, and cause the display device to display a live stream data and a three-dimensional point cloud and/or mesh and/or stereoscopic format, any other formats from the medical devices and/or a live surgery scene and/or from a camera mounted on a headset of a second device that is worn by a surgeon and other people to provide users with a real-life example of surgical techniques and live patient view. In some implementations, further comprising a reader from the memory of content, the reader being operably coupled to the microprocessor. In some implementations, the exchange of the second data with the medical application further comprises exchange the second data with a visceral surgery application. In some implementations, the exchange of the second data with the medical application further comprises exchanging the second data with a general surgery plan application wherein the general surgery plan application further comprises a resection planning and volume calculation application in visceral surgery (hepatectomy) to enable users to plan surgeries and calculate a volume of tissue that needs to be removed. In some implementations, wherein the resection planning and volume calculation features are specific to a medical field. In some implementations, the medical devices further comprise an ultrasound medical imaging device. In some implementations, the medical devices further comprise a laparoscopic surgical device. In some implementations, the display device further comprises first and/or second display device further comprises a XR (Extended reality device) such as mixed reality smartglasses device like HoloLens or virtual reality smartglasses like Meta Quest or the combination like Apple Vision Pro and other providers. In some implementations, the calendar entry is retrieved through an online scheduling system. In some implementations, the dynamically segmented files are downloaded from a secure server to the memory. In some implementations, the medical devices include devices from Medtronic. In some implementations, the three-dimensional point cloud of the live surgery is obtained through a video recording. In some implementations, interaction between the operators includes voice and/or video communication.

An apparatus operable to conduct a virtual medical masterclass that includes a microprocessor being adapted to, read a calendar entry from a memory, the calendar entry including time/date of a session and an identification of at least one participant involved in the session, retrieve from the memory a plurality of dynamically segmented files and other data of a plurality of patient cases, display on a display device the plurality of patient cases in three-dimensions, the plurality of patient cases being displayed as segmented objects and as volume with natural/cinematic rendering, exchange a first data to and from medical devices made by different manufacturers of the medical devices to provide a more realistic experience, change a status of operators to allowed interaction with each other to discuss cases and share knowledge, exchange a second data with a medical application, and display on the display device a live stream data and a three-dimensional point cloud and/or mesh and/or stereoscopic format, any other formats from the medical devices and/or a live surgery scene and/or from a camera mounted on a headset of a second device that is worn by a surgeon and other people to provide users with a real-life example of surgical techniques and live patient view. In some implementations, the microprocessor being further adapted to read content from the memory. In some implementations, the exchange of the second data with the medical application further comprises exchange the second data with a visceral surgery application. In some implementations, the exchange of the second data with the medical application further comprises exchanging the second data with a general surgery plan application wherein the general surgery plan application further comprises a resection planning and volume calculation application in visceral surgery (hepatectomy) to enable users to plan surgeries and calculate a volume of tissue that needs to be removed. In some implementations, the resection planning and volume calculation features are specific to a medical field. In some implementations, the medical devices further comprise an ultrasound medical imaging device. In some implementations, the medical devices further comprise a laparoscopic surgical device. In some implementations, the display device further comprises first and/or second display device further comprises a XR (Extended reality device) such as mixed reality smartglasses device like HoloLens or virtual reality smartglasses like Meta Quest or the combination like Apple Vision Pro and other providers. In some implementations, the calendar entry is retrieved through an online scheduling system. In some implementations, the dynamically segmented files are downloaded from a secure server to the memory. In some implementations, the medical devices include devices from Medtronic. In some implementations, the three-dimensional point cloud of the live surgery is obtained through a video recording. In some implementations, interaction between the operators includes voice and/or video communication.

1. A method of conducting a virtual medical masterclass, the method comprising:
reading from a memory a calendar entry, the calendar entry including a time/date of a session and an identification verification of a unique identifier number of at least one or more participant(s) involved in the session;
reading from the memory a plurality of customizing settings describing a virtual visual experience capturing a surgical environment and content distributed via auditory and visual channels from the memory or delivered by faculty experts as live feed;
retrieving from the memory a plurality of dynamically segmented patient files and other data of a plurality of patient cases;
displaying on a first display device the plurality of patient cases in three-dimensions, the plurality of patient cases with medical images being displayed as segmented objects, X-ray, CT, MRI, and any other volume with natural/cinematic rendering;
exchanging a first data to and from medical devices made by different manufacturers of the medical devices to provide a more realistic experience;
changing status of operators to allowed interaction with each other to discuss and evaluate predefined medical case objectives and collaborate to evaluate attainment of objectives in a virtual environment and remotely;
exchanging a second data with a medical application; and
displaying on the first display device a live stream data and a three-dimensional point cloud and/or mesh and/or stereoscopic format, any other formats from the medical devices and/or a live surgery scene and/or from a camera mounted on a headset of a second display device that is worn by a surgeon and other people to provide users with a real-life example of surgical techniques and live patient view, wherein the first display device and/or the second display device further comprises a XR (Extended reality) device.

2. The method of claim 1, further comprising reading from the memory a content.

3. The method of claim 1, wherein exchanging the second data with the medical application further comprises exchanging the second data with a specific medical application.

4. The method of claim 1, wherein the exchanging the second data with the medical application further comprises exchanging the second data with a resection planning and volume calculation application to enable users to plan surgeries and calculate a volume of tissue that needs to be removed, wherein the second data further comprises resection planning and volume calculation features that are specific to a medical field.

5. The method of claim 1, wherein the medical devices further comprise an ultrasound medical imaging device, HDMI from a surgical robot or fluoroscopy or microscope or X-ray.

6. The method of claim 1, wherein the medical devices further comprise a laparoscopic surgical device.

7. The method of claim 1, wherein the XR (Extended reality) device further comprises reality smartglasses.

8. The method of claim 1, wherein the calendar entry is retrieved through an online scheduling system.

9. The method of claim 1, wherein the dynamically segmented files are downloaded from a secure server to the memory.

10. The method of claim 1, wherein the medical devices include devices from medical device manufacturers.

11. The method of claim 1, wherein the three-dimensional point cloud of the live surgery is obtained through a video recording.

12. The method of claim 1, wherein interaction between the operators includes voice and/or video communication.

13. An apparatus operable to conduct a virtual medical masterclass, the apparatus comprising:
a microprocessor;
a reader of a calendar entry from a memory to retrieve from the memory a time/date of a session and to retrieve from the memory an identification of at least one participant involved in the session, the reader being operably coupled to the microprocessor;
a retriever from the memory a plurality of dynamically segmented files and other data of a plurality of patient cases, the retriever being operably coupled to the microprocessor;
a first display device operable to display the plurality of patient cases in three-dimensions, the plurality of patient cases being displayed as segmented objects and as volume with natural/cinematic rendering, the display device being operably coupled to the microprocessor;
an exchanger of a first data to and from medical devices made by different manufacturers of the medical devices to provide a more realistic experience, the exchanger of the first data being operably coupled to the microprocessor;
a changer of a status of operators to allowed interaction with each other to discuss cases and share knowledge, the changer being operably coupled to the microprocessor;
an exchanger of a second data with a medical application, the exchanger of the second data being operably coupled to the microprocessor;
the display device being further operable to display a live stream data and a three-dimensional point cloud of a live surgery and/or mesh and/or stereoscopic format, any other formats from the medical devices and/or a live surgery scene and/or from a camera mounted on a headset of a second device that is worn by a surgeon; and
the display device further operable to display live stream data from the medical devices,
wherein the first display device and/or the second display device further comprises a XR (Extended reality) device.

14. The apparatus of claim 13, further comprising a reader from the memory of content, the reader being operably coupled to the microprocessor.

15. The apparatus of claim 13, wherein the exchanger of the second data with the medical application further comprises an exchanger of the second data with a visceral surgery application.

16. The apparatus of claim 13, wherein the exchanger of the second data with the medical application further comprises an exchanger of the second data with a general surgery plan application wherein the general surgery plan application further comprises a resection planning and volume calculation application in visceral surgery (hepatectomy) to enable users to plan surgeries and calculate a volume of tissue that needs to be removed.

17. The apparatus of claim 16, wherein resection planning and volume calculation features are specific to a medical field.

18. The apparatus of claim 13, wherein the medical devices further comprise an ultrasound medical imaging device.

19. The apparatus of claim 13, wherein the medical devices further comprise a laparoscopic surgical device.

20. The apparatus of claim 13, wherein the XR (Extended reality) device further comprises a virtual reality smartglasses.
